(19) United States
(12) Patent Application Publication
(10) Pub. No.: US 2016/0256049 A1
(43) Pub. Date: Sep. 8, 2016

(54) METHOD OF ANALYSIS OF BODY REACTIONS TO EXTERNAL STIMULI

(71) Applicant: DECYLION SP. Z O.O., Warszawa (PL)

(72) Inventor: MARCIN GROCHOWINA, RZESZÓW (PL)

(73) Assignee: DECYLION SP. Z O.O., WARSZAWA (PL)

(21) Appl. No.: 14/433,940

(22) PCT Filed: Jul. 24, 2014
(86) PCT No.: PCT/PL2014/000088

§ 371 Date: Apr. 7, 2015

(30) Foreign Application Priority Data
Jun. 29, 2014 (PL) P.408690

(51) Int. Cl. A61B 3/11; A61B 3/00; A61B 5/00; A61B 3/14

(52) U.S. Cl. CPC A61B 3/112; A61B 3/145; A61B 3/0025; A61B 3/0008; A61B 5/0013; A61B 5/7282; A61B 2576/00

(57) ABSTRACT

A method of analysis of body reactions to external stimuli with the use of a microprocessor diagnostic system is characterized in that the image obtained from the camera (1) is analyzed in a microprocessor system (2), wherein the image is obtained by exposure of the eye to visible light emitted by an illuminator, and then a video sequence is registered, and the analysis is performed by the microprocessor (2) executing a program that implements an algorithm of image analysis for detection of characteristic features of the image which allow its identification, and in the first place the eye (3) is detected, and then, in the region of its pupil (4), geometric parameters (5) of the pupil and their changes over time are measured, and on the basis of the data obtained a diagnosis module (6) generates a test result (7) transmitted to a database (8).

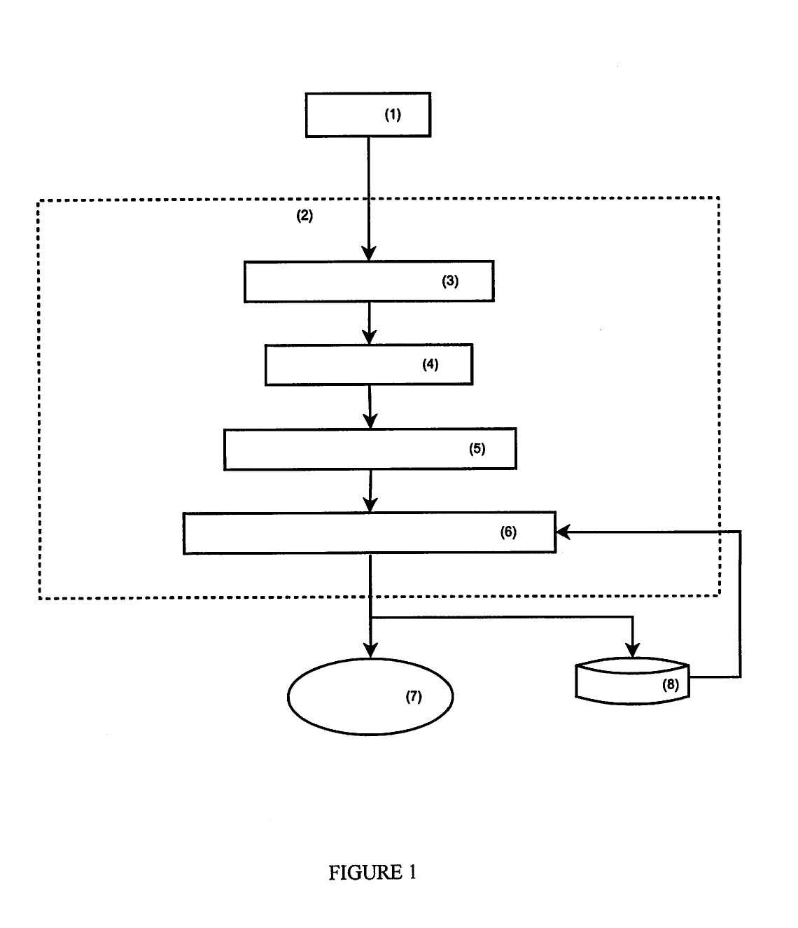

The object of the invention is a method of analysis of body reactions to external stimuli consisting in the use of a remotely controlled microprocessor diagnostic system, and in particular with the use of a mobile phone.

The problem of easy access to psychoactive agents which often can not be clearly classified in legal terms (e.g. designer drugs) grows in the modern world and poses serious problems not only of social nature but above all constitutes an educational challenge for tutors of young people. An important issue is the ability to quickly detect whether a suspected person is under the influence of narcotic drugs or stimulants. For this purpose, publicly available chemical tests reacting to the presence of the very drugs or their metabolic agents in body fluids, such as blood, saliva and urine, can be used.
However, selectivity of the above tests is problematic—each of them reacts to one particular agent, moreover, there may be problems with obtaining the material to be tested, particularly when the test requires blood sampling. When it is not possible to perform a chemical test, methods for assessing reaction to external stimuli can be used, but in this case the assessment is subjective and based on the experience of the person examining—lack of experience can lead to false conclusions.

From patent specification PL204415, a medical system for drug administration used for drug administration to a patient is known, the system comprising a medical device located at a first location, an electronic processor coupled to the medical device, a sensor coupled to the processor, and a remote controller located at a second location, situated away from the first location, wherin the remote controller has an input device used to control the operation of the electronic processor, and the sensor receives one or more signals, and transmits them to the processor, these signals can be obtained on the basis of the physiological condition of the patient and/or the environment in which the patient stays, and the processor receives the signals and performs a signal conversion. Based on the result of the calculation, the processor regulates the process of drug administration to the patient over time.

The aim of the invention is the use of objective methods of analysis of body reactions to external stimuli. Irritation of the eye's retina with light having an increased intensity results, under normal conditions, in pupillary constriction, wherein the time needed by the nervous system to perform this operation fluctuates around 1 second.

The essence of the invention consists in that the image obtained from the camera is analyzed in a microprocessor system, wherein the image is obtained by exposure of the eye to visible light emitted by an illuminator, and then a video sequence is registered, and the analysis is performed by a microprocessor executing a program that implements an algorithm of image analysis for detection of characteristic features of the image which allow its identification, and in the first place the eye is detected, and then, in the region of its pupil, geometric parameters of the pupil and their changes over time are measured, and on the basis of the data obtained a diagnosis module generates a test result.

In the case the body is under the influence of psychoactive agents, the reaction time may be prolonged or the reaction can be completely eliminated, and the pupil remains all the time expanded or constricted. Apart from confirming the influence of psychoactive agents, the type of reaction allows rough determination of a group of agents acting on the body.

The object of the invention is illustrated as an embodiment in the FIGURE in the form of a block diagram.

The image obtained from the camera 1 is analyzed in a microprocessor system 2, wherein the image is obtained by exposure of the eye to visible light emitted by an illuminator, and then a video sequence is registered, and the analysis is performed by the microprocessor executing a program that implements an algorithm of image analysis for detection of characteristic features of the image which allow its identification, and in the first place the eye 3 is detected, and then, in the region of its pupil 4, geometric parameters 5 of the pupil and their changes over time are measured, and on the basis of the data obtained a diagnosis module 6 generates a test result 7 transmitted to a database 8.
In the case the body is under the influence of psychoactive agents, the reaction time may be prolonged or the reaction can be completely eliminated, and the pupil remains all the time expanded or constricted. Apart from confirming the influence of psychoactive agents, the type of reaction allows rough determination of a group of agents acting on the body.

1. A method of analysis of body reactions to external stimuli with the use of a microprocessor diagnostic system, characterized in that the image obtained from the camera (1) is analyzed in a microprocessor system (2), wherein the image is obtained by exposure of the eye to visible light emitted by an illuminator, and then a video sequence is registered, and the analysis is performed by the microprocessor (2) executing a program that implements an algorithm of image analysis for detection of characteristic features of the image which allow its identification, and in the first place the eye (3) is detected, and then, in the region of its pupil (4), geometric parameters (5) of the pupil and their changes over time are measured, and afterwards on the basis of the data obtained, a diagnosis module (6) generates a test result (7) transmitted to a database (8).
